(19) United States
(12) Patent Application Publication
(10) Pub. No.: US 2004/0116453 A1
(43) Pub. Date: Jun. 17, 2004

(54) Synthesis and methods of use pyrimidine analogues and derivatives

(76) Inventors: David B. Fick, Newport Beach, CA (US); Mark M. Foreman, Tustin, CA (US); Alvin J. Glasky, Tustin, CA (US)

Correspondence Address: Attn: Louis C. Cullman, STRADLING YOCCA CARLSON & RAUTH, Suite 1600, 660 Newport Center Drive, Newport Beach, CA, 92660, US

(21) Appl. No.: 10/648,046

(22) Filed: Aug. 25, 2003

Related U.S. Application Data
(63) Continuation of application No. 09/907,273, filed on Jul. 17, 2001, now abandoned.

(51) Int. Cl. A61K 31/505; C 07D 4 3/02

(52) U.S. Cl. 514/275; 544/330; 544/331

(57) ABSTRACT

A pyrimidine derivative or analogue comprises an amino-substituted six-membered heterocyclic moiety, moiety A, linked through a linker L to a moiety B, where B is a carboxylic acid, a carboxylic acid ester, or a moiety of the structure N(Y₁)-D, where Y₁ can be one of a variety of substituents, including hydrogen or alkyl, and D is a moiety that enhances the pharmacological effects, promotes absorption, or promotes blood-brain barrier penetration of the derivative or analogue. The moiety A can have two or three nitrogen atoms in the ring; typically, the moiety A is a pyrimidine moiety, with two nitrogen atoms in the ring. The moiety B can be one of a variety of moieties, including moieties having nootropic activity or other biological or physiological activity.

### BACKGROUND OF THE INVENTION

1. Field of the Invention

The present invention is directed to pyrimidine derivatives and analogues, particularly pyrimidine derivatives or analogues in which the pyrimidine derivative or analogue is covalently linked to another moiety to form a bifunctional conjugate.

2. General Background and State of the Art

Diseases and degenerative conditions of the central nervous system are among the most severe, long-lasting, and chronic diseases and conditions affecting man. Although much research has been done on such diseases and conditions, effective treatment remains elusive. These diseases and conditions include Alzheimer's disease, Huntington's disease, amyotrophic lateral sclerosis (Lou Gehrig's disease), Parkinson's disease, multiple sclerosis, stroke, and other neurodegenerative disorders, which may be genetic, spontaneous or drug-induced.

There is therefore a need for improved compounds and methods for treating such conditions. The need for such improved compounds and methods has been increased by the discovery that such compounds are capable of increasing neuronal function, stimulating nerve growth or regeneration and can act through the induction of neurotrophic factors such as nerve growth factor, NT-3, brain-derived neurotrophic factor (BDNF), or ciliary neurotrophic factor (CNTF). Such compounds may stimulate nerve regeneration or neurogenesis in the peripheral nervous system or central nervous system, or neuroprotection, and may therefore be of use in the treatment of the diseases and conditions referred to above.

Most of the attention given to such compounds has been focused upon compounds in which one component is a purine or purine analogue. However, pyrimidines also play an important role in cellular metabolism. For example, most eukaryotic organisms have specific enzymes that methylate thymine in DNA where it is used as a marker. In addition, the pyrimidine-derived molecule uridine diphosphate glucose (UDPG) plays an important role in sugar metabolism. Accordingly, increased attention is being focused upon bifunctional compounds in which one of the components is a pyrimidine or a pyrimidine analogue. However, such molecules have not been explored nearly as thoroughly as their purine-derived counterparts. However, increasing attention is being paid to these pyrimidine analogues in view of the biological activities of pyrimidines.

There is therefore a particular need for the development of additional compounds that have improved activity in stimulating neuronal function, regeneration, neurogenesis, and that have neuroprotective activity. There is further a need for compounds that have activities that provide treatment for or relief from symptoms of diseases and conditions such as Alzheimer's disease, Huntington's disease, Parkinson's disease, multiple sclerosis, stroke and other neurodegenerative disorders, which may be genetic, spontaneous or drug-induced. Examples of these symptoms include reduced cognition, emotional control, and sensory or motor function. There is a particular need for the development of new compounds that have improved bioavailability. There is a further need for compounds with a greater degree of activity as measured by a dose-response curve assay and for compounds with a different spectrum of activities.

### INVENTION SUMMARY

One aspect of the present invention is pyrimidine derivatives and analogues. In general, a pyrimidine derivative or analogue of the present invention has the schematic structure:

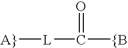

where:

(1) A is an amino-substituted six-membered heterocyclic moiety of formula (I)

where:

(a) if the bond between N₁ and C₆ is a single bond, then the bond between C₆ and R₆ is a double bond, R₆ is O or S, and R₁ is hydrogen, alkyl, aralkyl, cycloalkyl, or heteroaralkyl;

(b) if the bond between N₁ and C₆ is a double bond, then the bond between C₆ and R₆ is a single bond, R₁ is not present, and R₆ is hydrogen, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6- membered ring which can contain 1 other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(c) if the bond between C₂ and N₃ is a single bond, then the bond between C₂ and R₂ is a double bond, R₂ is O or S, and R₃ is hydrogen or alkyl;

(d) if the bond between C₂ and N₃ is a double bond, then the bond between C₂ and R₂ is a single bond, R₃ is not present, and R₂ is hydrogen, alkyl, aralkyl, cycloalkyl, heteroaralkyl, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain 1 other heteroatom which can be N, O, or S, of which the N can be further substituted with Y₃, where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(e) R₄ is hydrogen, alkyl, aralkyl, heteroaralkyl, alkanoyl, aroyl, heteroaroyl, aralkanoyl, heteroaralkanoyl, alkylsulfonyl, arylsulfonyl, heteroarylsulfonyl, alkoxycarbonyl, aryloxycarbonyl, heteroaryloxycarbonyl, alkylaminocarbonyl, arylaminocarbonyl, or heteroarylaminocarbonyl;

(f) A₅ is carbon or nitrogen;

(g) if A₅ is nitrogen, then R₅ is not present;

(h) if A₅ is carbon, then R₅ is hydrogen, amino, alkyl, alkoxy, halo, nitro, aryl, cyano, alkenyl, or aralkyl;

(i) if R₅ and R₆ are present together, and are alkyl, they can be taken together to form a 5- or 6-membered ring which can contain 1 other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(j) N₄ is bonded to L;

(2) L is a hydrocarbyl moiety of 1 to 6 carbon atoms that can be cyclic, with the hydrocarbyl moiety being optionally substituted with one or more substituents selected from the group consisting of lower alkyl, amino, hydroxy, lower alkoxy, lower alkylamino, lower alkylthio and oxo; and

(3) B is —OZ or N(Y₁)-D, where Z is hydrogen, alkyl, aryl, heteroaryl, cycloalkyl, aralkyl, or heteroaralkyl, D is a moiety that promotes absorption of the derivative or analogue, and Y₁ is hydrogen, alkyl, aryl, heteroaryl, aralkyl, or heteroaralkyl, which, when taken with D, can form a cyclic 5- or 6-membered saturated structure which can contain one other heteroatom which can be O, N, or S, of which N can be further substituted with Y₄, where Y₄ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

Typically, A is a pyrimidine moiety with an additional nitrogen substituent at N₄ which, in turn, is bonded to the linker L. The pyrimidine moiety can be a naturally-occurring or synthetic pyrimidine moiety.

B is either: (i) a moiety with the structure —OZ, where Z is hydrogen, alkyl, aryl, heteroaryl, cycloalkyl, aralkyl, or heteroaralkyl; or (ii) a moiety with the structure —N(Y₁)-D, where D is a moiety that promotes absorption of the derivative or analogue that can be substituted as indicated above.

If B is a moiety with the structure —OZ, it is a carboxylic acid or a carboxylic acid ester. Typically, if B is a carboxylic acid ester, the moiety Z is one of methyl, ethyl, propyl, butyl, or isobutyl. More typically, Z is hydrogen or ethyl.

If B is a moiety with the structure —N(Y₁)-D, Y₁ is hydrogen, alkyl, aryl, heteroaryl, aralkyl, or heteroaralkyl, which, when taken with D, can form a cyclic 5- or 6-membered saturated ring which can contain one other heteroatom which can be O, N, or S, of which N can be further substituted with Y₅, where Y₅ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, heteroaralkylaminocarbonyl, in which the alkyl portions can be cyclic and can contain from 1 to 3 heteroatoms which can be N, O, or S. Typically, Y₁ is hydrogen or lower alkyl. Most typically, Y₁ is hydrogen.

Typically, the pyrimidine derivative or analogue has a logP of from about 1 to about 4 to enhance bioavailability and central nervous system (CNS) penetration. Using this guideline, one of ordinary skill in the art can choose the appropriate moieties B for a particular moiety A in order to ensure the bioavailability and CNS penetration of a pyrimidine analogue or derivative according to the present invention. For example, if a highly hydrophobic moiety A is chosen, with particularly hydrophobic substituents on the pyrimidine moiety, then a more hydrophilic moiety B can be used.

In one alternative, B is a moiety containing at least one carboxyl, carboxamide, carboxyl ester, or carbonyl function.

In another alternative, B is a cyclic or acyclic moiety containing at least one hydroxyl, primary amino, secondary amino, tertiary amino, sulfhydryl, or sulfonamidyl function.

Particular examples of pyrimidine derivatives and analogues according to the present invention include: (1) 4-[3-(2-amino-6-chloropyrimidin-4-ylamino)propionylamino] benzoic acid ethyl ester; (2) 4-[3-(6-chloropyrimidin-4-ylamino)propionylamino] benzoic acid ethyl ester; and (3) 4-[3-(5-amino-6-chloropyrimidin-4-ylamino)propionylamino] benzoic acid ethyl ester.

Another aspect of the present invention is methods of use of the pyrimidine derivatives and analogues described above. One aspect of a method of use of pyrimidine derivatives and analogues according to the present invention is a method of stimulating neuronal function such as improved cognition, involving neuronal regeneration or axo-dendritic complexity in the central and peripheral nervous systems comprising the step of administering an effective amount of a pyrimidine derivative or analogue according to the present invention to the mammal. Another aspect of a method of use of pyrimidine derivatives and analogues according to the present invention is a method of stimulating neuronal function such as improved cognition, involving initiating neurogenesis in the central nervous system of a mammal comprising the step of administering an effective amount of a pyrimidine derivative or analogue according to the present invention to the mammal. Yet another aspect of a method of use of pyrimidine derivatives and analogues according to the present invention is a method of stimulating neuronal function involving mechanism associated with neuroprotection in the central or peripheral nervous system of a mammal comprising the step of administering an effective amount of a pyrimidine derivative or analogue according to the present invention to the mammal.

Other methods according to the present invention include a method of stimulating neuronal function involving either inhibition of the formation of the amyloid beta-peptide (Aβ) or stimulating the formation of the secreted derivative of the amyloid precursor protein known as sAPPα by administering to a patient with a neurological disease or a patient at risk of developing a neurological disease an effective quantity of a pyrimidine derivative or analogue according to the present invention.

Another aspect of the present invention is pharmaceutical compositions. A pharmaceutical composition according to the present invention comprises: (1) an effective amount of a pyrimidine derivative or analogue according to the present invention; and (2) a pharmaceutically acceptable carrier.

### DETAILED DESCRIPTION OF THE PREFERRED EMBODIMENTS

I. Pyrimidine Derivatives and Analogues

One aspect of the present invention is pyrimidine derivatives and analogues.

In its most general aspect, a pyrimidine derivative or analogue according to the present invention has the schematic structure:

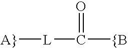

where:

(1) A is an amino-substituted six-membered heterocyclic moiety of formula (I)

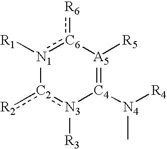

where:

(a) if the bond between N₁ and C₆ is a single bond, then the bond between C₆ and R₆ is a double bond, R₆ is O or S, and R₁ is hydrogen, alkyl, aralkyl, cycloalkyl, or heteroaralkyl;

(b) if the bond between N₁ and C₆ is a double bond, then the bond between C₆ and R₆ is a single bond, R₁ is not present, and R₆ is hydrogen, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6- membered ring which can contain 1 other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(c) if the bond between C₂ and N₃ is a single bond, then the bond between C₂ and R₂ is a double bond, R₂ is O or S, and R₃ is hydrogen or alkyl;

(d) if the bond between C₂ and N₃ is a double bond, then the bond between C₂ and R₂ is a single bond, R₃ is not present, and R₂ is hydrogen, alkyl, aralkyl, cycloalkyl, heteroaralkyl, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain 1 other heteroatom which can be N, O, or S, of which the N can be further substituted with Y₃, where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;

(e) R₄ is hydrogen, alkyl, aralkyl, heteroaralkyl, alkanoyl, aroyl, heteroaroyl, aralkanoyl, heteroaralkanoyl, alkylsulfonyl, arylsulfonyl, heteroarylsulfonyl, alkoxycarbonyl, aryloxycarbonyl, heteroaryloxycarbonyl, alkylaminocarbonyl, arylaminocarbonyl, or heteroarylaminocarbonyl;

(f) A₅ is carbon or nitrogen;

(g) if A₅ is nitrogen, then R₅ is not present;

(h) if A₅ is carbon, then R₅ is hydrogen, amino, alkyl, alkoxy, halo, nitro, aryl, cyano, alkenyl, or aralkyl;

(i) N₄ is bonded to L;

(2) L is a hydrocarbyl moiety of 1 to 6 carbon atoms that can be cyclic, with the hydrocarbyl moiety being optionally substituted with one or more substituents selected from the group consisting of lower alkyl, amino, hydroxy, lower alkoxy, lower alkylamino, lower alkylthio and oxo; and

(3) B is —OZ or N(Y₁)-D, where Z is hydrogen, alkyl, aryl, heteroaryl, cycloalkyl, aralkyl, or heteroaralkyl, D is a moiety that promotes absorption of the derivative or analogue, and Y₁ is hydrogen, alkyl, aryl, heteroaryl, aralkyl, or heteroaralkyl, which, when taken with D, can form a cyclic 5- or 6-membered saturated structure which can contain one other heteroatom which can be O, N, or S, of which N can be further substituted with Y₄, where Y₄ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

Typically, Y₁ is hydrogen or lower alkyl. Most typically, Y₁ is hydrogen.

Typically, the pyrimidine derivative or analogue has a logP of from about 1 to about 4 to enhance bioavailability and central nervous system (CNS) penetration. Using this guideline, one of ordinary skill in the art can choose the appropriate moieties B for a particular moiety A in order to ensure the bioavailability and CNS penetration of a pyrimidine analogue or derivative according to the present invention. For example, if a highly hydrophobic moiety A is chosen, with particularly hydrophobic substituents on the pyrimidine moiety, then a more hydrophilic moiety B can be used.

In many examples, in a pyrimidine analogue or derivative according to the present invention, the moiety B has a biological, physiological, or pharmacological function, and the pyrimidine analogue or derivative is referred to as a “bifunctional conjugate.” However, it is not required in pyrimidine analogues or derivatives according to the present invention that the moiety B has a biological, physiological, or pharmacological function. The moiety B can serve as a carrier to improve bioavailability or to optimize the physical characteristics of the molecule without having a separate biological function, physiological function, or pharmacological function.

In many pyrimidine analogues or derivatives according to the present invention, the moiety B includes a p-aminobenzoic acid, a p-aminobenzoic acid ester, a m-aminobenzoic acid, or a m-aminobenzoic acid ester. However, the moiety B can include other groups.

Typically, the moiety A is a pyrimidine moiety in which A₅ is carbon so that the ring of the moiety A has two nitrogen atoms in the positions in which they are present in pyrimidines. The pyrimidine moiety can be variously substituted so that it has the structure of a naturally-occurring pyrimidine such as thymine, uracil, cytosine, or another naturally-occurring or synthetic pyrimidine. Particularly preferred examples of pyrimidines are discussed below. When A is a pyrimidine moiety, the result is a pyrimidine derivative; when A is other than a pyrimidine moiety, then the result is a pyrimidine analogue.

When A is a pyrimidine moiety, typical pyrimidine moieties include, but are not limited to, cytosine, thymine, uracil, 3-methyluracil, 3-methylthymine, 4-methylcytosine, 5-methylcytosine, 5-hydroxymethylcytosine, 5-hydroxyuracil, 5-carboxymethyluracil, 5-hydroxymethyluracil, 2-thiouracil, 5-methylamino-2-thiouracil, 5-methyl-2-thiouracil, 2-thiocytosine, 2-aminopyrimidinone, 2-amino-4-chloropyrimidine, 4-chloropyrimidine, 5-amino-4-chloropyrimidine, 4-chloro-5-methylpyrimidine, 4-chloro-5-hydroxymethylpyrimidine, 5-carboxymethyl-4-chloropyrimidine, or pyrimidinone. Other pyrimidine moieties can be used. The numbering of these pyrimidines as separate molecules as recited herein is the conventional numbering in which the numbering proceeds clockwise from N₁, which is at the bottom of the formula as conventionally depicted. The numbering of positions in the pyrimidine moieties of conjugates according to the present invention is as depicted in Formula (I), above. Other unsubstituted or substituted pyrimidine moieties can be used.

In one preferred alternative, R₂ is O and R₃ is hydrogen. In one preferred embodiment of this alternative, R₅ is hydrogen, R₆ is amino, and the pyrimidine moiety is cytosine. In another preferred embodiment of this alternative,. R₁ is hydrogen, R₅ is methyl, R₆ is O, and the pyrimidine moiety is thymine. In another preferred embodiment of this alternative, R₁ is hydrogen, R₅ is hydrogen, R₆ is O, and the pyrimidine moiety is uracil. In another preferred embodiment of this alternative, R₁ is methyl, R₅ is hydrogen, R₆ is O, and the pyrimidine moiety is 3-methyluracil. In another preferred embodiment of this alternative, R₁ is methyl, R₅ is methyl, R₆ is O, and the pyrimidine moiety is 3-methylthymine. In another preferred embodiment of this alternative, R₅ is hydrogen, R₆ is methylamino, and the pyrimidine moiety is 4-methylcytosine. In another preferred embodiment of this alternative, R₅ is methyl, R₆ is amino, and the pyrimidine moiety is 5-methylcytosine. In another preferred embodiment of this alternative, R₅ is hydroxymethyl, R₆ is amino, and the pyrimidine moiety is 5-hydroxymethylcytosine. In another preferred embodiment of this alternative, R₅ is hydroxyl, R₆ is O, and the pyrimidine moiety is 5-hydroxyuracil. In another preferred embodiment of this alternative, R₅ is carboxymethyl, R₆ is O, and the pyrimidine moiety is 5-carboxymethyluracil. In another preferred embodiment of this alternative, R₅ is hydroxymethyl, R₆ is O, and the pyrimidine moiety is 5-hydroxymethyluracil.

In another preferred alternative, R₂ is S and R₃ is hydrogen. In one preferred embodiment of this alternative, R₁ is hydrogen, R₅ is hydrogen, R₆ is O, and the pyrimidine moiety is 2-thiouracil. In another preferred embodiment of this alternative, R₁ is hydrogen, R₅ is methylamino, R₆ is O, and the pyrimidine moiety is 5-methylamino-2-thiouracil. In another preferred embodiment of this alternative, R₁ is hydrogen, R₅ is methyl, R₆ is O, and the pyrimidine moiety is 5-methyl-2-uracil. In another preferred embodiment of this alternative, R₅ is hydrogen, R₆ is amino, and the pyrimidine moiety is 2-thiocytosine.

In another preferred alternative, R₂ is amino and the bond between C₂ and N₃ is a double bond. In one preferred embodiment of this alternative, R₁ is hydrogen, R₅ is hydrogen, R₆ is O, and the pyrimidine moiety is 2-aminopyrimidinone. In another preferred embodiment of this alternative, R₅ is hydrogen, R₆ is Cl, and the pyrimidine moiety is 2-amino-4-chloropyrimidine.

In another preferred alternative, R₂ is hydrogen and the bond between C₂ and N₃ is a double bond. In one preferred embodiment of this alternative, R₅ is hydrogen, R₆ is Cl, and the pyrimidine moiety is 4-chloropyrimidine. In another preferred embodiment of this alternative, R₅ is amino, R₆ is Cl, and the pyrimidine moiety is 4-chloropyrimidine. In another preferred embodiment of this alternative, R₅ is methyl, R₆ is Cl, and the pyrimidine moiety is 4-chloro-5-methylpyrimidine. In another preferred embodiment of this alternative, R₅ is hydroxymethyl, R₆ is Cl, and the pyrimidine moiety is 4-chloro-5-hydroxymethylpyrimidine. In another preferred embodiment of this alternative, R₅ is carboxymethyl, R₆ is Cl, and the pyrimidine moiety is 5-carboxymethyl-4-chloropyrimidine. In another preferred embodiment of this alternative, R, is hydrogen, methyl, or ethyl, R₅ is hydrogen, methyl, or ethyl, and R₆ is O. Most preferably, in this embodiment, R₁ is hydrogen, R₅ is hydrogen, and the pyrimidine moiety is pyrimidinone.

The linker L is described further below. L is a hydrocarbyl moiety of 1 to 6 carbon atoms that can be cyclic, with the hydrocarbyl moiety being optionally substituted with one or more substituents selected from the group consisting of lower alkyl, amino, hydroxy, lower alkoxy, lower alkylamino, lower alkythio, and oxo.

In accordance with the present invention, and as used herein, the following terms, when appearing alone or as part of a moiety including other atoms or groups, are defined with the following meanings, unless explicitly stated otherwise. In addition, all groups described herein can be optionally substituted unless such substitution is excluded. The term “alkyl,” as used herein at all occurrences, refers to saturated aliphatic groups including straight-chain, branched-chain, and cyclic groups, all of which can be optionally substituted. Preferred alkyl groups contain 1 to 10 carbon atoms. Suitable alkyl groups include methyl, ethyl, and the like, and can be optionally substituted. The term “alkenyl,” as used herein at all occurrences, refers to unsaturated groups which contain at least one carbon-carbon double bond and includes straight-chain, branched-chain, and cyclic groups, all of which can be optionally substituted. Preferable alkenyl groups have 2 to 10 carbon atoms. The term “alkoxy” refers to the ether —O-alkyl, where alkyl is defined as above. The term “aryl” refers to aromatic groups which have at least one ring having a conjugated π-electron system and includes carbocyclic aryl and biaryl, both of which may be optionally substituted. Preferred aryl groups have 6 to 10 carbon atoms. The term “aralkyl” refers to an alkyl group substituted with an aryl group. Suitable aralkyl groups include benzyl and the like; these groups can be optionally substituted. The term “aralkenyl” refers to an alkenyl group substituted with an aryl group. The term “heteroaryl” refers to carbon-containing 5-14 membered cyclic unsaturated radicals containing one, two, three, or four O, N, or S heteroatoms and having 6, 10, or 14 π-electrons delocalized in one or more rings, e.g., pyridine, oxazole, indole, thiazole, isoxazole, pyrazole, pyrrole, each of which can be optionally substituted as discussed above. The term “sulfonyl” refers to the group —S(O₂)—. The term “alkanoyl” refers to the group —C(O)Rg, where Rg is alkyl. The term “aroyl” refers to the group —C(O)Rg, where Rg is aryl. Similar compound radicals involving a carbonyl group and other groups are defined by analogy. The term “aminocarbonyl” refers to the group —NHC(O)—. The term “oxycarbonyl” refers to the group —OC(O)—. The term “heteroaralkyl” refers to an alkyl group substituted with a heteroaryl group. Similarly, the term “heteroaralkenyl” refers to an alkenyl group substituted with a heteroaryl group. As used herein, the term “lower,” in reference to an alkyl or the alkyl portion of an another group including alkyl, is defined as a group containing one to six carbon atoms. The term “optionally substituted” refers to one or more substituents that can be lower alkyl, aryl, amino, hydroxy, lower alkoxy, aryloxy, lower alkylamino, arylamino, lower alkylthio, arylthio, or oxo, in some cases, other groups can be included, such as cyano, acetoxy, or halo. The term “halo” refers generally to fluoro, chloro, bromo, or iodo; more typically, “halo” refers to chloro.

A preferred linker has the structure —(CH₂)ₙ— wherein n is an integer from 1 to 6. As detailed below, for most preferred embodiments of pyrimidine derivatives or analogues according to the present invention, a preferred linker has n equal to 2 or 3. Particular examples of pyrimidine derivatives or analogues according to the present invention follow.

A number of pyrimidine derivatives or analogues according to the present invention are optically active, owing to the presence of chiral carbons or other centers of asymmetry. In cases where pyrimidine derivatives or analogues according to the present invention are optically active, all of the possible enantiomers or diastereoisomers are included unless otherwise indicated despite possible differences in activity.

Particularly preferred pyrimidine moieties for the moiety A include 2-amino-4-chloropyrimidine, 4-chloropyrimidine, and 5-amino-4-chloropyrimidine.

In addition to these examples of moieties suitable as moiety A, other moieties can serve as moiety A, including moieties with nitrogen at A₅ or substituents at N₄.

In general, pyrimidine derivatives and analogues that are within the scope of the present invention also include salts and prodrug esters of these pyrimidine derivatives and analogues. It is well known that organic compounds, including pyrimidines and other components of these pyrimidine derivatives and analogues have multiple groups that can accept or donate protons, depending upon the pH of the solution in which they are present. These groups include carboxyl groups, hydroxyl groups, amino groups, sulfonic acid groups, and other groups known to be involved in acid-base reactions. The recitation of a pyrimidine derivative or analogue according to the present invention includes such salt forms as occur at physiological pH or at the pH of a pharmaceutical composition unless specifically excluded.

Similarly, prodrug esters can be formed by reaction of either a carboxyl or a hydroxyl group on the pyrimidine derivative or analogue with either an acid or an alcohol to form an ester. Typically, the acid or alcohol includes a lower alkyl group such as methyl, ethyl, propyl, isopropyl, butyl, isobutyl, and tertiary butyl. These groups can be substituted with substituents such as hydroxy, halo, or other substituents. Such prodrugs are well known in the art and need not be described further here. The prodrug is converted into the active compound by hydrolysis of the ester linkage, typically by intracellular enzymes. Other suitable groups that can be used to form prodrug esters are well known in the art.

As indicated above, the linker L is a hydrocarbyl moiety of 1 to 6 carbon atoms that can be cyclic, with the hydrocarbyl moiety being optionally substituted with one or more substituents selected from the group consisting of lower alkyl, amino, hydroxy, lower alkoxy, lower alkylamino, lower alkylthio, and oxo. Preferably, the linker L has the structure —(CH₂)ₙ— wherein n is an integer from 1 to 6. As detailed below, for most preferred embodiments of pyrimidine derivatives or analogues according to the present invention, a preferred linker has n equal to 2 or 3.

The moiety B is either: (i) —OZ, where Z is hydrogen, alkyl, aryl, heteroaryl, cycloalkyl, aralkyl, or heteroaralkyl; or (ii) N(Y₁)-D, where D is a moiety that promotes absorption of the derivative or analogue, and Y₁ is hydrogen, alkyl, aryl, heteroaryl, aralkyl, heteroaralkyl, which, when taken with D, can form a cyclic 5- or 6-membered saturated ring which can contain one other heteroatom which can be O, N, or S, of which N can be further substituted with Y₄, where Y₄ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, heteroaralkylaminocarbonyl, in which the alkyl portions can be cyclic and can contain from 1 to 3 heteroatoms which can be N, O, or S. Typically, Y₁ is hydrogen. Where the moiety B is —OZ, the moiety B is a carboxylic acid or carboxylic acid or ester. Typically, where B is a carboxylic acid ester, the moiety Z is a lower alkyl, such as methyl, ethyl, butyl, propyl, or isopropyl.

In one alternative, the moiety D, as described above, is a moiety having at least one polar, charged, or hydrogen-bond-forming group to improve the metabolic and bioavailability properties of the pyrimidine derivative or analogue. The moiety D can be, but is not limited to, a moiety with physiological or biological activity such as nootropic activity. In one alternative, the moiety D can be a moiety containing at least one carboxyl, carboxamide, carboxyl ester, or carbonyl function. In another alternative, the moiety D can be a moiety containing at least one hydroxyl, primary amino, secondary amino, tertiary amino, sulfhydryl, or sulfonamidyl function. The moiety D can be cyclic or acyclic. Preferred examples of the moiety D are described below.

When the moiety D is a cyclic or acyclic moiety containing at least one carbonyl, carboxamide, carboxyl ester, or carbonyl function, in one preferred example, D is a carboxylic acid or carboxylic acid ester with the structure

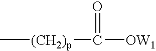

wherein p is an integer from 1 to 6 and W₁ is selected from the group consisting of hydrogen and lower alkyl. Typically, if W₁ is lower alkyl, it is methyl, ethyl, propyl, butyl, or isobutyl. Typically, p is 3. Typically, W₁ is hydrogen or ethyl.

In another preferred example, D and Y₁ are taken together to form a piperazine derivative as described in D. Manetti et al., “Molecular Simplification of 1,4-Diazabicyclo[4.3.0]nonan-9-ones Gives Piperazine Derivatives That Maintain High Nootropic Activity,” J. Med. Chem. 43: 4499-4507 (“Manetti et al. (2000)”). B is an analogue of structure

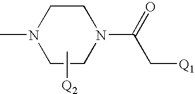

wherein Q₁ is hydrogen, methyl, ethyl, butyl, or propyl, and Q₂ is hydrogen or methyl, where, if Q₂ is methyl, it can be located at either of the two possible positions in the piperazine ring.

In another preferred example, D has the structure

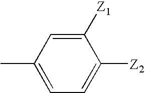

where one of Z₁ and Z₂ is hydrogen, and the other of Z₁ and Z₂ is —COOH or —COOW₁, wherein W₁ is alkyl. Typically, W₁ is selected from the group consisting of methyl, ethyl, propyl, butyl, and isobutyl. Either of Z₁ or Z₂ can be hydrogen. When Z₁ is hydrogen and Z₂ is —COOH, the moiety B is p-aminobenzoic acid (PABA). When Z₁ is —COOH and Z₂ is hydrogen, the moiety B is m-aminobenzoic acid (MABA). When Z₁ is hydrogen and Z₂ is —COOW₁, the moiety B is an ester of p-aminobenzoic acid (PABA). When Z₁ is —COOW₁ and Z₂ is hydrogen, the moiety B is an ester of m-aminobenzoic acid (MABA). Typically, these esters are ethyl esters.

When the moiety D is a moiety that contains at least one hydroxyl, primary amino, secondary amino, tertiary amino, sulfhydryl, or sufonamidyl function, in one preferred example, D is a phenylsulfonamidyl moiety of structure

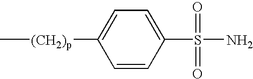

wherein p is an integer from 0 to 6. Typically, p is 2.

In another preferred example, D is an alkylpyridyl moiety of structure

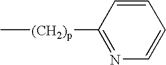

wherein p is an integer from 1 to 6. Typically, p is 1.

In another preferred example, D is a dialkylaminoalkyl moiety of the structure

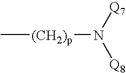

wherein p is an integer from 1 to 6 and Q₇ and Q₈ are alkyl, aralkyl, heteroaralkyl, aryl, heteroaryl, alkanoyl, aroyl, aralkanoyl, heteroaralkanoyl, or heteroaroyl in which the alkyl portions can be cyclic and can contain from 1 to 3 heteroatoms which can be N, O, or S, and when Q₇ and Q₈ are present together and are alkyl, they can be taken together to form a 5 or 6 member ring which may contain 1 other heteroatom which can be N, O, or S, of which the N may be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

Where Q₇ and Q₈ can be taken together to form a five or six member ring, the ring is typically pyrrolidine, piperidine, or morpholine. The pyrrolidine ring can be optionally substituted with oxo. The piperidine ring can be optionally substituted with methyl or ethyl. Typically, p is 2 or 3.

In another preferred example, D is an alkylpyrrolidine moiety of the structure

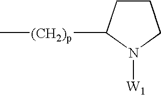

wherein p is an integer from 1 to 6 and W₁ is selected from the group consisting of methyl, ethyl, and propyl. Typically, W₁ is methyl. Typically, p is 2.

Preferably, a pyrimidine analogue or derivative according to the present invention has a logP of from about 1 to about 4 in order to optimize bioavailability and CNS penetration of the pyrimidine analogue or derivative.

In general, any moiety A can be combined with any linker L and any moiety B, including the appropriate moiety D, to produce a pyrimidine analogue or derivative according to the present invention. However, there exist a number of particularly preferred pyrimidine analogues or derivatives according to the present invention. These include the following:

(1) 4-[3-(2-amino-6-chloropyrimidin-4-ylamino)propionylamino] benzoic acid ethyl ester (Example 1);

(2) 4-[3-(6-chloropyrimidin-4-ylamino)propionylamino] benzoic acid ethyl ester (Example 2);

(3) 4-[3-(5-amino-6-chloropyrimidin-4-ylamino)propionylamino] benzoic acid ethyl ester (Example 3);

(4) 4-[3-(2-amino-6-chloropyrimidin-4-ylamino)propionylamino] benzoic acid;

(5) 4-[3-(6-chloropyrimidin-4-ylamino)propionylamino] benzoic acid;

(6) 4-[3-(5-amino-6-chloropyrimidin-4-ylamino)propionylamino] benzoic acid ethyl ester;

(7) 3-[3-(2-amino-6-chloropyrimidin-4-ylamino)propionylamino] benzoic acid ethyl ester;

(8) 3-[3-(6-chloropyrimidin-4-ylamino)propionylamino] benzoic acid ethyl ester;

(9) 3-[3-(5-amino-6-chloropyrimidin-4-ylamino)propionylamino] benzoic acid ethyl ester;

(10) 3-[3-(2-amino-6-chloropyrimidin-4-ylamino)propionylamino] benzoic acid;

(11) 3-[3-(6-chloropyrimidin-4-ylamino)propionylamino] benzoic acid; and

(12) 3-[3-(5-amino-6-chloropyrimidin-4-ylamino)propionylamino] benzoic acid ethyl ester.

II. Methods of Synthesis of Pyrimidine Derivatives and Analogues According to the Present Invention

Methods for synthesis of pyrimidine derivatives and analogues according to the present invention are modified from those described, for example, in U.S. Pat. No. 5,091,432 to Glasky, incorporated herein by this reference. In one general route in which the pyrimidine derivatives and analogues according to the present invention contain a substituted or unsubstituted 4-chloropyrimidine moiety, the pyrimidine moiety is substituted with a linker which in turn is linked to the moiety B that completes the molecule as described above. This route comprises the steps of: (1) synthesizing an appropriately substituted pyrimidine moiety linked to an aliphatic linker in which the linker is terminated with a carboxyl group protected such as with an alkyl ester; (2) hydrolyzing the alkyl ester (or other analogous protecting group) to yield a carboxylic acid; (3) activating the free carboxylic acid by converting it to a nitrophenyl ester or acid chloride; (4) reacting the nitrophenyl ester or acid chloride with an appropriate group that can form an amide or other stable covalent linkage with the carboxyl moiety, with appropriate protection for the moiety reacting with the ester if required; and (5) hydrolyzing the protective group protecting the moiety reacting with the ester to produce the final product.

The length of the aliphatic linker covalently bound to the pyrimidine moiety can be varied to vary the distance between the pyrimidine moiety and the moiety B in the pyrimidine derivative or analogue.

Another route comprises the steps of: (1) synthesizing an appropriate moiety B containing the linker terminally substituted with an amine, (2) reacting this amine with an appropriately substituted 4,6-dichloropyrimidine to produce the final product.

Examples of this chemistry may be found in (1) Dang, et al “A new regio-defined synthesis of PMEA.” Nucleosides and Nucleotides 17: 1445-1452 (1998), (2) Gibson, et al “Specific inhibitors in vitamin biosynthesis. Part 10. Synthesis of 7- and 8-substituted 7-deazaguanines.” J. Chem. Soc. Perkin Trans.118: 3025-3032 (1998).

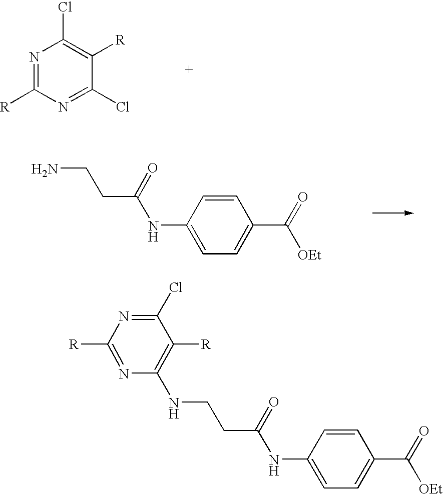

III. Methods of Use of Pyrimidine Derivatives and Analogues According to the Present Invention

One aspect of a method of use of pyrimidine derivatives and analogues according to the present invention is a method of stimulating regeneration of a mammalian neuron in the peripheral nervous system of a mammal comprising the step of administering an effective amount of a pyrimidine derivative or analogue according to the present invention to the mammal.

Another aspect of a method of use of pyrimidine derivatives and analogues according to the present invention is a method of stimulating neurogenesis in the central nervous system of a mammal comprising the step of administering an effective amount of a pyrimidine derivative or analogue according to the present invention to the mammal.

Yet another aspect of a method of use of pyrimidine derivatives and analogues according to the present invention is a method of stimulating neuroprotection in the central or peripheral nervous system of a mammal comprising the step of administering an effective amount of a pyrimidine derivative or analogue according to the present invention to the mammal.

Exemplary dosages in accordance with the teachings of the present invention for these pyrimidine derivatives and analogues range from 0.0001 mg/kg to 60 mg/kg, though alternative dosages are contemplated as being within the scope of the present invention. Suitable dosages can be chosen by the treating physician by taking into account such factors as the size, weight, age, and sex of the patient, the physiological state of the patient, the severity of the condition for which the pyrimidine derivative or analogue is being administered, the response to treatment, the type and quantity of other medications being given to the patient that might interact with the pyrimidine derivative or analogue, either potentiating it or inhibiting it, and other pharmacokinetic considerations such as liver and kidney function. In general, purine derivatives and analogues according to the present invention have a minimal effective dose of 10 mg/kg i.p. or less.

The administration of pyrimidine derivatives or analogues according to the present invention is believed to increase the level of mRNA encoding at least one neurotrophic factor that can affect the growth, differentiation, survival, or functioning of neurons in the peripheral or central nervous systems.

The neurotrophic factor can be one of nerve growth factor, NT-3, brain-derived neurotrophic factor (BDNF), or ciliary neurotrophic factor (CNTF); the neurotrophic factor can also be another neurotrophic factor as are well known in the art.

Although Applicants do not intend to be bound by this theory, the increase in the level of mRNA of one or more of these neurotrophic factors brought about by methods according to the present invention employing pyrimidine derivatives or analogues according to the present invention is believed to promote neuronal survival.

The term “effective amount” as used herein in this specification means an amount of the pyrimidine derivative or analogue that causes a detectable increase in the messenger RNA level of at least one of the recited neurotrophic factors or of another neurotrophic factor known in the art that can be measured. Methods of measuring the mRNA levels typically involve hybridization to probes containing mRNA-specific sequences and detecting the quantity of hybrid nucleic acid formed. The hybrid nucleic acid formed is typically detected by a label incorporated in one of the two nucleic acid strands forming the hybrid. This label can be radioactive or non-radioactive; if non-radioactive, it can be fluorescent, chemiluminescent, bioluminescent, enzymatic, or can make use of another detectable property. Detection can also be performed using the polymerase chain reaction (PCR) mechanism or a variant thereof. PCR is described in detail in U.S. Pat. No. 4,683,195 to Mullis et al. and U.S. Pat. No. 4,683,202 to Mullis et al. Other detection methods, including other amplification methods, are known in the art. One particularly suitable method uses reverse transcription with MMLV reverse transcriptase followed by PCR.

Another method employing pyrimidine derivatives and analogues according to the present invention is a method of increasing neuronal function by either inhibiting the formation of the amyloid beta-peptide (Aβ) or stimulating the formation of the secreted derivative of the amyloid precursor protein known as sAPPα by administering to a patient with a neurological disease or a patient at risk of developing a neurological disease an effective quantity of a pyrimidine derivative or analogue according to the present invention as described above. The neurological disease can be a neurodegenerative disease, such as, but not limited to, Alzheimer's disease (AD). Alternatively, the neurological disease can be a neurodevelopmental disorder such as, but not limited to, Down's Syndrome.

Yet another aspect of methods according to the present invention is a method of treating peripheral neuropathy comprising administering to a patient with peripheral neuropathy an effective quantity of a pyrimidine derivative or analogue according to the .present invention. Typically, in this method, the administration of the pyrimidine derivative or analogue induces peripheral nerve sprouting in the skin of the patient to whom the pyrimidine derivative or analogue is administered. The peripheral nerve sprouting can be nociceptive nerve sprouting. Typically, the nociceptive nerve sprouting is induced without the occurrence of hyperalgesia. The peripheral neuropathy can be diabetic neuropathy or can be a neuropathy associated with the following conditions: acromegaly, hypothyroidism, AIDS, leprosy, Lyme disease, systemic lupus erythematosus, rheumatoid arthritis, Sjögren's Syndrome, periarteritis nodosa, Wegener's granulomatosis, cranial arteritis, sarcoidosis or the administration of other therapeutic agents, e.g. oncolytic drugs.

Yet another aspect of the present invention is a method of increasing neuronal function by inducing proliferation of neuronal precursor cells. In general, the method comprises administering to a mammal an effective quantity of a pyrimidine derivative or analogue according to the present invention as described above to induce proliferation of neuronal precursor cells in the mammal.

Yet another aspect of the present invention is a method of increasing neuronal function by inducing differentiation of neuronal precursor cells. In general, the method comprises administering to a mammal an effective quantity of a pyrimidine derivative or analogue according to the present invention as described above to induce differentiation of neuronal precursor cells in the mammal.

Depending upon the particular needs of the individual subject involved, the pyrimidine derivative or analogue according to the present invention may be administered in various doses to provide effective treatment concentrations based upon the teachings of the present invention. What constitutes an effective amount of the selected pyrimidine derivative or analogue will vary based upon such factors as the activity of the selected pyrimidine derivative or analogue, the physiological characteristics of the subject, the extent or nature of the subject's disease or condition, and the method of administration. Generally, initial doses will be modified to determine the optimum dosage for treatment of the particular mammalian subject. The pyrimidine derivative or analogues can be administered using a number of different routes including orally, topically, transdermally, intraperitoneal injection, or intravenous injection directly into the bloodstream. Of course, effective amounts of the pyrimidine derivative or analogue can also be administered through injection into the cerebrospinal fluid or infusion directly into the brain, if desired.

The methods of the present invention can be effected using a pyrimidine derivative or analogue according to the present invention administered to a mammalian subject either alone or in combination as a pharmaceutical formulation. Further, the pyrimidine derivative or analogue according to the present invention can be combined with pharmaceutically acceptable excipients and carrier materials such as inert solid diluents, aqueous solutions, or non-toxic organic solvents. If desired, these pharmaceutical formulations can also contain preservatives and stabilizing agents and the like, as well as minor amounts of auxiliary substances such as wetting or emulsifying agents, as well as pH buffering agents and the like which enhance the effectiveness of the active ingredient. The pharmaceutically acceptable carrier can be chosen from those generally known in the art including, but not limited to, human serum albumin, ion exchangers, dextrose, alumina, lecithin, buffer substances such as phosphate, glycine, sorbic acid, propylene glycol, polyethylene glycol, and salts or electrolytes such as protamine sulfate, sodium chloride, or potassium chloride. Other carriers can be used.

Liquid compositions can also contain liquid phases either in addition to or to the exclusion of water. Examples of such additional liquid phases are glycerin, vegetable oils such as cottonseed oil, organic esters such as ethyl oleate, and water-oil emulsions.

The compositions can be made into aerosol formations (i.e., they can be “nebulized”) to be administered via inhalation. Aerosol formulations can be placed into pressurized acceptable propellants, such as dichloromethane, propane, or nitrogen. Other suitable propellants are known in the art.

Formulations suitable for parenteral administration, such as, for example, by intravenous, intramuscular, intradermal, and subcutaneous routes, include aqueous and non-aqueous isotonic sterile injection solutions. These can contain antioxidants, buffers, preservatives, bacteriostatic agents, and solutes that render the formulation isotonic with the blood of the particular recipient. Alternatively, these formulations can be aqueous or non-aqueous sterile suspensions that can include suspending agents, thickening agents, solublizers, stabilizers, and preservatives. Compositions suitable for use in methods according to the present invention can be administered, for example, by intravenous infusion, orally, topically, intraperitoneally, intravesically, or intrathecally. Formulations of pyrimidine derivative or analogues suitable for use in methods according to the present invention can be presented in unit-dose or multi-dose sealed containers, in physical forms such as ampules or vials.

IV. Pharmaceutical Compositions

Another aspect of the present invention is pharmaceutical compositions. A pharmaceutical composition according to the present invention comprises: (1) an effective amount of a pyrimidine derivative or analogue according to the present invention as described above; and (2) a pharmaceutically acceptable carrier.

A pharmaceutically acceptable carrier can be chosen from those generally known in the art including, but not limited to, human serum albumin, ion exchangers, alumina, lecithin, buffer substances such as phosphates, glycine, sorbic acid, potassium sorbate, and salts or electrolytes such as potassium sulfate. Other carriers can be used.

The invention is illustrated by the following Examples. These Examples are presented for illustrative purposes only and are not intended to limit the invention.

### EXAMPLE 1

### Synthesis of 4-[3-(2-Amino-6-chloropyrimidin-4-ylamino)propionylamino] Benzoic Acid Ethyl Ester

The starting materials 4-(3-aminopropionylamino)benzoic acid ethyl ester (500 mg, 2.1 mmol), 2-mino-4,6-dichloropyrimidine (590 mg, 3.6 mmol), and triethylamine (590 μL, 4.2 mmol) were combined in ethanol (25 mL) and heated to reflux for 10 hours. After cooling to room temperature, a precipitate formed and was isolated via vacuum filtration. The solid was identified as the excess starting 2-amino-4,6-dichloropyrimidine. Water (100 mL) was then added to the filtrate and another precipitate formed and was also isolated via vacuum filtration washing with water. The isolated solid was dried under vacuum to obtain 710 mg (93%) of the title compound.

### EXAMPLE 2

### Synthesis of 4-[3-(6-Chloropyrimidin-4-ylamino)propionylamino] Benzoic Acid Ethyl Ester

The pyrimidine 4-[3-(6-chloropyrimidin-4-ylamino)propionylamino] benzoic acid ethyl ester was synthesized by the route used in Example 1 except that 4,6-dichloropyrimidine replaced the 2-amino-4,6-dichloropyrimidine.

### EXAMPLE 3

### Synthesis of 4-[3-(5-Amino-6-chloropyrimidin-4-ylamino)propionylamino] Benzoic Acid Ethyl Ester

The pyrimid ine 4-[3-(5-amino-6-chloropyrimidin-4-ylamino)propionylamino] benzoic acid ethyl ester was synthesized by the route used in Example 1 except that 5-amino-4,6-dichloropyrimidine replaced the 2-amino-4,6-dichloropyrimidine.

### EXAMPLE 4

### Passive Avoidance Method of Testing Compounds

Passive avoidance is an acute memory paradigm in which mice are allowed to enter a dark compartment from a lighted compartment, but are given a footshock (2 mA for 5 seconds) when they enter the dark compartment. Twenty-four hours after this training session, animals that are placed back in the lighted compartment of two compartment (light-dark) apparatus do not make the transition into the dark compartment. If an amnestic agent (30 mg/kg cycloheximide i.p. in saline) immediately after the training session is given to the animals, they will make the transition into the dark compartment (i.e memory of the shock is lost). Compounds with suspected nootropic or anti-amnestic effects are given by i.p. administration two.hours prior to the training trial in attempt to block the effects of cycloheximide. Mice that exhibit positive nootropic effects are those that avoid moving into the dark chamber. This behavioral response is defined as passive avoidance. A no effect response in this test is defined as a failure to stay in the lighted compartment for 120 seconds. All compounds cited here have nootropic or antiamnestic activity at doses of 10 mg/kg i.p. or less.

Advantages of the Present Invention

The present invention provides pyrimidine analogues and derivatives that exert a number of biological and physiological functions, particularly increased neuronal function that may involve nerve regeneration in the peripheral nervous system, neurogenesis in the central nervous system, and neuroprotection. The pyrimidine analogues and derivatives of the present invention are capable of passing through the blood-brain barrier and exerting their effects in the central nervous system. The components of the pyrimidine analogue or derivative can be chosen to optimize the desired activity or range of activities of the molecule and to increase bioavailability.

Although the present invention has been described in considerable detail, with reference to certain preferred versions thereof, other versions and embodiments are possible. Therefore, the scope of the invention is determined by the following claims.

We claim:

1. A pyrimidine derivative or analogue having the schematic structure: 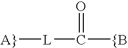
where:
(a) A is an amino-substituted six-membered heterocyclic moiety of formula (I) 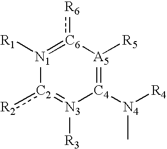
where:
(i) if the bond between N₁ and C₆ is a single bond, then the bond between C₆ and R₆ is a double bond, R₆ is O or S, and R₁ is hydrogen, alkyl, aralkyl, cycloalkyl, or heteroaralkyl;
(ii) if the bond between N₁ and C₆ is a double bond, then the bond between C₆ and R₆ is a single bond, R₁ is not present, and R₆ is hydrogen, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain 1 other heteroatom which can be N, O, or S, of which the N can be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(iii) if the bond between C₂ and N₃ is a single bond, then the bond between C₂ and R₂ is a double bond, R₂ is O or S, and R₃ is hydrogen or alkyl;
(iv) if the bond between C₂ and N₃ is a double bond, then the bond between C₂ and R₂ is a single bond, R₃ is not present, and R₂ is hydrogen, alkyl, aralkyl, cycloalkyl, heteroaralkyl, halo, amino, OQ₁, SQ₁, NHNH₂, NHOQ₁, NQ₁Q₂, or NHQ₁, where Q₁ and Q₂ are alkyl, aralkyl, heteroaralkyl, aryl, heteroaryl, alkanoyl, aroyl, aralkanoyl, heteroaralkanoyl, heteroaroyl, alkylsulfonyl, arylsulfonyl, heteroarylsulfonyl, aralkylsulfonyl, or heteroaralkylsulfonyl in which the alkyl portions can be cyclic and can contain from 1 to 3 heteroatoms which can be N, O, or S, and when Q₁ and Q₂ are present together and are alkyl, they can be taken together to form a 5- or 6-membered ring which can contain 1 other heteroatom which can be N, O, or S, of which the N can be further substituted with Y₃, where Y₃ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S;
(v) R₄ is hydrogen, alkyl, aralkyl, heteroaralkyl, alkanoyl, aroyl, heteroaroyl, aralkanoyl, heteroaralkanoyl, alkylsulfonyl, arylsulfonyl, heteroarylsulfonyl, alkoxycarbonyl, aryloxycarbonyl, heteroaryloxycarbonyl, alkylaminocarbonyl, arylaminocarbonyl, or heteroarylaminocarbonyl;
(vi) A₅ is carbon or nitrogen;
(vii) if A₅ is nitrogen, then R₅ is not present;
(viii) if A₅ is carbon, then R₅ is hydrogen, amino, alkyl, alkoxy, halo, nitro, aryl, cyano, alkenyl, or aralkyl;
(x) (ix) N₄ is bonded to L;
(b) L is a hydrocarbyl moiety of 1 to 6 carbon atoms that can be cyclic, with the hydrocarbyl moiety being optionally substituted with one or more substituents selected from the group consisting of lower alkyl, amino, hydroxy, lower alkoxy, lower alkylamino, lower alkylthio and oxo; and
(c) B is —OZ or N(Y₁)-D, where Z is hydrogen, alkyl, aryl, heteroaryl, cycloalkyl, aralkyl, or heteroaralkyl, D is a moiety that promotes absorption of the derivative or analogue, and Y₁ is hydrogen, alkyl, aryl, heteroaryl, aralkyl, or heteroaralkyl, which, when taken with D, can form a cyclic 5- or 6-membered saturated structure which can contain one other heteroatom which can be O, N, or S, of which N can be further substituted with Y₄, where Y₄ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

2. The pyrimidine derivative or analogue of claim 1 wherein A₅ is carbon and the six-membered heterocyclic moiety is a pyrimidine moiety.

3. The pyrimidine derivative or analogue of claim 2 wherein R₂ is O and R₃ is hydrogen.

4. The pyrimidine derivative or analogue of claim 3 wherein R₅ is hydrogen, R₆ is amino, and the pyrimidine moiety is cytosine.

5. The pyrimidine derivative or analogue of claim 3 wherein R₁ is hydrogen, R₅ is methyl, R₆ is O, and the pyrimidine moiety is thymine.

6. The pyrimidine derivative or analogue of claim 3 wherein R₁ is methyl, R₅ is hydrogen, R₆ is O, and the pyrimidine moiety is uracil.

7. The pyrimidine derivative or analogue of claim 3 wherein R₁ is hydrogen, R₅ is methyl, R₆ is O, and the pyrimidine moiety is 3-methyluracil.

8. The pyrimidine derivative or analogue of claim 3 wherein R₁ is methyl, R₅ is methyl, R₆ is O, and the pyrimidine moiety is 3-methylthymine.

9. The pyrimidine derivative or analogue of claim 3 wherein R₅ is hydrogen, R₆ is methylamino, and the pyrimidine moiety is 4-methylcytosine.

10. The pyrimidine derivative or analogue of claim 3 wherein R₅ is methyl, R₆ is amino, and the pyrimidine moiety is 5-methylcytosine.

11. The pyrimidine derivative or analogue of claim 3 wherein R₅ is hydroxymethyl, R₆ is amino, and the pyrimidine moiety is 5-hydroxymethylcytosine.

12. The pyrimidine derivative or analogue of claim 3 wherein R₁ is hydrogen, R₅ is hydroxyl, R₆ is O, and the pyrimidine moiety is 5-hydroxyuracil.

13. The pyrimidine derivative or analogue of claim 3 wherein R₁ is hydrogen, R₅ is carboxymethyl, R₆ is O, and the pyrimidine moiety is 5-carboxymethyluracil.

14. The pyrimidine derivative or analogue of claim 3 wherein R₁ is hydrogen, R₅ is hydroxymethyl, R₆ is O, and the pyrimidine moiety is 5-hydroxymethyluracil.

15. The pyrimidine derivative or analogue of claim 2 wherein R₂ is S and R₃ is hydrogen.

16. The pyrimidine derivative or analogue of claim 15 wherein R₁ is hydrogen, R₅ is hydrogen, R₆ is O, and the pyrimidine moiety is 2-thiouracil.

17. The pyrimidine derivative or analogue of claim 15 wherein R₁ is hydrogen, R₅ is methylamino, R₆ is O, and the pyrimidine moiety is 5-methylamino-2-thiouracil.

18. The pyrimidine derivative or analogue of claim 15 wherein R₁ is hydrogen, R₅ is methyl, R₆ is O, and the pyrimidine moiety is 5-methyl-2-thiouracil.

19. The pyrimidine derivative or analogue of claim 15 wherein R₅ is hydrogen, R₆ is amino, and the pyrimidine derivative or analogue is 2-thiocytosine.

20. The pyrimidine derivative or analogue of claim 2 wherein R₂ is amino and the bond between C₂ and N₃ is a double bond.

21. The pyrimidine derivative or analogue of claim 20 wherein R₁ is hydrogen, R₅ is hydrogen, R₆ is O, and the pyrimidine moiety is 2-aminopyrimidinone.

22. The pyrimidine derivative or analogue of claim 20 wherein R₅ is hydrogen, R₆ is Cl, and the pyrimidine moiety is 2-amino-4-chloropyrimidine.

23. The pyrimidine derivative or analogue of claim 2 wherein R₂ is hydrogen and the bond between C₂ and N₃ is a double bond.

24. The pyrimidine derivative or analogue of claim 23 wherein R₅ is hydrogen, R₆ is Cl, and the pyrimidine moiety is 4-chloropyrimidine.

25. The pyrimidine derivative or analogue of claim 23 wherein R₅ is amino, R₆ is Cl, and the pyrimidine moiety is 5-amino-4-chloropyrimidine.

26. The pyrimidine derivative or analogue of claim 23 wherein R₅ is methyl, R₆ is Cl, and the pyrimidine moiety is 4-chloro-5-methylpyrimidine.

27. The pyrimidine derivative or analogue of claim 23 wherein R₅ is hydroxymethyl, R₆ is Cl, and the pyrimidine moiety is 4-chloro-5-hydroxymethylpyrimidine.

28. The pyrimidine derivative or analogue of claim 23 wherein R₅ is carboxymethyl, R₆ is Cl, and the pyrimidine moiety is 4-chloro-5-carboxymethylpyrimidine.

29. The pyrimidine derivative or analogue of claim 23 wherein R₁ is hydrogen, methyl, or ethyl, R₅ is hydrogen, methyl, or ethyl, and R₆ is O.

30. The pyrimidine derivative or analogue of claim 29 wherein R1 is hydrogen, R5 is hydrogen, and the pyrimidine moiety is pyrimidinone.

31. The pyrimidine derivative or analogue of claim 1 wherein L has the structure —(CH₂)ₙ— wherein n is an integer from 1 to 6.

32. The pyrimidine derivative or analogue of claim 31 wherein n is 2.

33. The pyrimidine derivative or analogue of claim 31 wherein n is 3.

34. The pyrimidine derivative or analogue of claim 1 wherein the moiety B is —OZ.

35. The pyrimidine derivative or analogue of claim 34 wherein Z is hydrogen.

36. The pyrimidine derivative or analogue of claim 34 wherein Z is alkyl.

37. The pyrimidine derivative or analogue of claim 36 wherein Z is selected from the group consisting of methyl, ethyl, butyl, propyl, and isopropyl.

38. The pyrimidine derivative or analogue of claim 1 wherein the moiety B is N(Y₁)-D.

39. The pyrimidine derivative or analogue of claim 38 wherein Y₁ is hydrogen.

40. The pyrimidine derivative or analogue of claim 38 wherein Y₁ is lower alkyl.

41. The pyrimidine derivative or analogue of claim 40 wherein Y₁ is methyl.

42. The pyrimidine derivative or analogue of claim 38 wherein D is a moiety having at least one polar, charged, or hydrogen-bond-forming group to increase the water-solubility of the pyrimidine derivative or analogue.

43. The pyrimidine derivative or analogue of claim 42 wherein D is a carboxylic acid or carboxylic acid ester with the structure 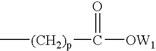
wherein p is an integer from 1 to 6 and W₁ is selected from the group consisting of hydrogen and lower alkyl.

44. The pyrimidine derivative or analogue of claim 43 wherein W₁ is hydrogen.

45. The pyrimidine derivative or analogue of claim 43 wherein W₁ is ethyl.

46. The pyrimidine derivative or analogue of claim 42 wherein D and Y₁ are taken together to form a piperazine derivative of the structure 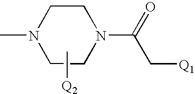
wherein Q₁ is hydrogen, methyl, ethyl, butyl, or propyl, and Q₂ is hydrogen or methyl, where, if Q₂ is methyl, it can be located at either of the two possible positions in the piperazine ring.

47. The pyrimidine derivative or analogue of claim 42 wherein D has the structure 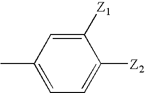
where one of Z₁ and Z₂ is hydrogen, and the other of Z₁ and Z₂ is —COOH or —COOW₁, wherein W₁ is alkyl.

48. The pyrimidine derivative or analogue of claim 47 wherein W₁ is selected from the group consisting of methyl, ethyl, propyl, butyl, and isobutyl.

49. The pyrimidine derivative or analogue of claim 42 wherein D is a phenylsulfonamidyl moiety of the structure 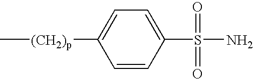
wherein p is an integer from 0 to 6.

50. The pyrimidine derivative or analogue of claim 42 wherein D is an alkylpyridyl moiety of structure 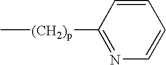
wherein p is an integer from 1 to 6.

51. The pyrimidine derivative or analogue of claim 42 wherein D is a dialkylaminoalkyl moiety of the structure 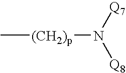
wherein p is an integer from 1 to 6 and Q₇ and Q₈ are alkyl, aralkyl, heteroaralkyl, aryl, heteroaryl, alkanoyl, aroyl, aralkanoyl, heteroaralkanoyl, or heteroaroyl in which the alkyl portions can be cyclic and can contain from 1 to 3 heteroatoms which can be N₁O, or S, and when Q₇ and Q₈ are present together and are alkyl, they can be taken together to form a 5 or 6 member ring which may contain 1 other heteroatom which can be N, O, or S, of which the N may be further substituted with Y₂, where Y₂ is alkyl, aryl, heteroaryl, aralkyl, heteroaralkyl, alkanoyl, aroyl, heteroaroyl, aralkanoyl, heteroaralkanoyl, alkylsulfonyl, arylsulfonyl, heteroarylsulfonyl, aralkylsulfonyl, heteroaralkylsulfonyl, alkoxycarbonyl, aryloxycarbonyl, heteroaryloxycarbonyl, aralkoxycarbonyl, heteroaralkoxycarbonyl, alkylaminocarbonyl, arylaminocarbonyl, heteroarylaminocarbonyl, aralkylaminocarbonyl, or heteroaralkylaminocarbonyl, in which the alkyl portions can be cyclic and can contain from 1 to 3 heteroatoms which can be N, O, or S.

52. The pyrimidine derivative or analogue of claim 51 wherein Q₇ and Q₈ are each alkyl.

53. The pyrimidine derivative or analogue of claim 52 wherein Q₇ and Q₈ are each selected from the group consisting of methyl, ethyl, propyl, and isopropyl.

54. The pyrimidine derivative or analogue of claim 52 wherein Q₇ and Q₈ are taken together to form a five- or six-membered optionally substituted ring.

55. The pyrimidine derivative or analogue of claim 54 wherein the ring is a morpholinyl ring.

56. The pyrimidine derivative or analogue of claim 54 wherein the ring is a pyrrolidinyl ring that is optionally substituted with oxo.

57. The pyrimidine derivative or analogue of claim 54 wherein the ring is a piperidinyl ring that is optionally substituted with methyl or ethyl.

58. The pyrimidine derivative or analogue of claim 42 wherein D is an alkylpyrrolidinyl moiety of the structure 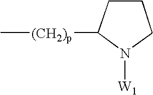
wherein p is an integer from 1 to 6 and W₁ is selected from the group consisting of methyl, ethyl, and propyl.

59. The pyrimidine derivative or analogue of claim 1 that has a logP of from about 1 to about 4.

60. A pyrimidine derivative or analogue that is 4-[3-(2-amino-6-chloropyrimidin-4-ylamino)propionylamino] benzoic acid ethyl ester.

61. A pyrimidine derivative or analogue that is 4-[3-(6-chloropyrimidin-4-ylamino)propionylamino] benzoic acid ethyl ester.

62. A pyrimidine derivative or analogue that is 4-[3-(5-amino-6-chloropyrimidin-4-ylamino)propionylamino] benzoic acid ethyl ester.

63. A pyrimidine derivative or analogue that is 4-[3-(2-amino-6-chloropyrimidin-4-ylamino)propionylamino] benzoic acid.

64. A pyrimidine derivative or analogue that is 4-[3-(6-chloropyrimidin-4-ylamino)propionylamino] benzoic acid.

65. A pyrimidine derivative or analogue that is 4-[3-(5-amino-6-chloropyrimidin-4-ylamino)propionylamino] benzoic acid.

66. A pyrimidine derivative or analogue that is 3-[3-(2-amino-6-chloropyrimidin-4-ylamino)propionylamino] benzoic acid ethyl ester.

67. A pyrimidine derivative or analogue that is 3-[3-(6-chloropyrimidin-4-ylamino)propionylamino] benzoic acid ethyl ester.

68. A pyrimidine derivative or analogue that is 3-[3-(5-amino-6-chloropyrimidin-4-ylamino)propionylamino] benzoic acid ethyl ester.

69. A pyrimidine derivative or analogue that is 3-[3-(2-amino-6-chloropyrimidin-4-ylamino)propionylamino] benzoic acid.

70. A pyrimidine derivative or analogue that is 3-[3-(6-chloropyrimidin-4-ylamino)propionylamino] benzoic acid.

71. A pyrimidine derivative or analogue that is 3-[3-(5-amino-6-chloropyrimidin-4-ylamino)propionylamino] benzoic acid.
